(19) United States
(12) Patent Application Publication
(10) Pub. No.: US 2023/0154010 A1
(43) Pub. Date: May 18, 2023

(54) IMAGE PROCESSING METHOD, IMAGE PROCESSING DEVICE, AND PROGRAM

(71) Applicant: NIKON CORPORATION, Tokyo (JP)

(72) Inventors: Mariko HIROKAWA, Yokohama-shi (JP); Yasushi TANABE, Fujisawa-shi (JP)

(73) Assignee: NIKON CORPORATION, Tokyo (JP)

(21) Appl. No.: 17/769,288

(22) PCT Filed: Oct. 18, 2019
(86) PCT No.: PCT/JP2019/041219

§ 371 Date: Apr. 14, 2022

(51) Int. Cl. G06T 7/194; G06T 7/11; G06T 7/00; A61B 3/12

(52) U.S. Cl. CPC G06T 7/194; G06T 7/11; G06T 7/0012; A61B 3/1241; A61B 3/1233; G06T 2207/20021; G06T 2207/30041; G06T 2207/10024; G06T 2207/30101; G06T 2207/10101

(57) ABSTRACT

A processor acquires a first fundus image of an examined eye including a foreground area and a background area other than the foreground area. The processor also generate a second fundus image by performing background processing to replace a first pixel value of a pixel configuring the background area with a second pixel value different from the first pixel value.

### TECHNICAL FIELD

The present invention relates to an image processing method, an image processing device, and a program.

### BACKGROUND ART

U.S. Pat. No. 7,445,337 discloses generating a fundus image in which a periphery of a fundus region (circular shape) is infilled in black as a background color, and displaying the fundus image on a display. Sometimes trouble such as mis-detection occurs when performing image processing of such a fundus image having an infilled periphery.

### SUMMARY OF INVENTION

An image processing method of a first aspect of the technology disclosed herein includes a processor acquiring a first fundus image of an examined eye including a foreground area and a background area other than the foreground area, and the processor generating a second fundus image by performing background processing to replace a first pixel value of a pixel configuring the background area with a second pixel value different from the first pixel value.

An image processing device of a second aspect of the technology disclosed herein includes a memory, and a processor coupled to the memory. The processor acquires a first fundus image of an examined eye including a foreground area and a background area other than the foreground area, and generates a second fundus image by performing background processing to replace a first pixel value of a pixel configuring the background area with a second pixel value different from the first pixel value.

A program of a third aspect of the technology disclosed herein causes a computer to execute processing including acquiring a first fundus image of an examined eye including a foreground area and a background area other than the foreground area, and generating a second fundus image by performing background processing to replace a first pixel value of a pixel configuring the background area with a second pixel value different from the first pixel value.

### BRIEF DESCRIPTION OF DRAWINGS

FIG. 1 is a block diagram of an ophthalmic system 100.

FIG. 2 is a schematic configuration diagram illustrating an overall configuration of an ophthalmic device 110.

FIG. 3 is a diagram illustrating a UWF RG color fundus image UWFGP obtained by imaging a fundus of an examined eye 12 with an ophthalmic device 110, and a fundus image (fundus camera image) FCGQ obtained by imaging the fundus of the examined eye 12 with a non-illustrated fundus camera.

FIG. 4 is a block diagram of configuration of an electrical system of a server 140.

FIG. 5 is a block diagram illustrating functionality of a CPU 262 of a server 140.

FIG. 6 is a flowchart illustrating an image processing program.

FIG. 7 is a flowchart of a retinal blood vessel removal processing program of step 300 of FIG. 6.

FIG. 8 is a flowchart illustrating a background infill processing program of step 302 of FIG. 6.

FIG. 9 is a flowchart illustrating a processing program to extract blood vessels of step 306 of FIG. 6.

FIG. 10A is a diagram illustrating a choroidal vascular image G1.

FIG. 10B is a diagram illustrating a background processing complete image G2.

FIG. 10C is a diagram illustrating a blood vessel emphasis image G3.

FIG. 10D is a diagram illustrating a blood vessel extraction image G4.

FIG. 11A is a diagram illustrating a choroidal vascular image G1 obtained with related technology.

FIG. 11B is a diagram illustrating a blood vessel emphasis image G7 obtained with related technology.

FIG. 11C is a diagram illustrating a threshold value image G8 obtained with related technology.

FIG. 11D is a diagram illustrating a blood vessel extraction image G9 obtained with conventional technology.

FIG. 12 is a diagram illustrating a foreground area FG, a background area BG, and a boundary BD in a choroidal vascular image G1.

FIG. 13A is a diagram of a Modified Example 1 of background infill processing and illustrates a way in which a pixel value of each of the pixels of the background area BG is transformed into a value of a pixel of the foreground area FG nearest in distance to the respective pixel.

FIG. 13B is a diagram schematically illustrating a foreground area FG and a background area BG in a choroidal vascular image G1.

FIG. 13C is a diagram of a Modified Example 2 of background infill processing and illustrates transforming a pixel value of each of the pixels of the background area BG using a value larger than the value of the respective pixel by a specific value.

FIG. 13D is a diagram of a Modified Example 3 of background infill processing and illustrates transforming a pixel value of each of the pixels of the background area BG using a value smaller than a value a pixel of the foreground area FG nearest in distance to the respective pixel by a specific value.

FIG. 13E is a diagram of a Modified Example 4 of background infill processing and illustrates transforming a pixel value of each of the pixels of the background area BG to an average value of all pixels of the foreground area FG.

FIG. 13F is a diagram of a Modified Example 5 of background infill processing and illustrates transforming a pixel value of each of the pixels of the background area BG so as to be gradually greater as a distance from a center CP of the foreground area FG increases.

FIG. 13G is a diagram of a Modified Example 6 of background infill processing and illustrates transforming a pixel value of each of the pixels of the background area BG so as to be gradually smaller as a distance from a center CP of the foreground area FG increases.

FIG. 14 is a diagram illustrating an examination screen 400A.

FIG. 15 is a diagram illustrating an examination screen 400B.

FIG. 16 is a diagram illustrating a combined image G14 obtained by overlaying a blood vessel extraction image G4 on an original fundus image (UWF RG color fundus image UWFGP).

FIG. 17 is a diagram illustrating emphasizing blood vessels by applying a frame to blood vessels bt.

FIG. 18 is a diagram illustrating a blurred image Gb obtained by blurring a blood vessel emphasis image G3.

### DESCRIPTION OF EMBODIMENTS

Detailed explanation follows regarding a first exemplary embodiment of the present invention, with reference to the drawings.

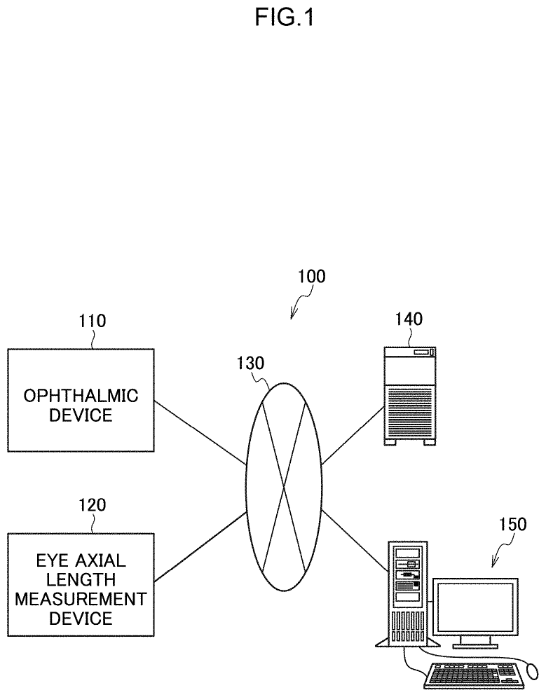

Explanation follows regarding a configuration of an ophthalmic system 100, with reference to FIG. 1. As illustrated in FIG. 1, the ophthalmic system 100 includes an ophthalmic device 110, an eye axial length measurement device 120, a management server device (referred to hereafter as “server”) 140, and an image display device (referred to hereafter as “viewer”) 150. The ophthalmic device 110 acquires an image of the fundus. The eye axial length measurement device 120 measures the axial length of the eye of a patient. The server 140 stores fundus images that were obtained by imaging the fundus of patients using the ophthalmic device 110 in association with patient IDs. The viewer 150 displays medical information such as fundus images acquired from the server 140.

The server 140 is an example of an “image processing device” of technology disclosed herein.

The ophthalmic device 110, the eye axial length measurement device 120, the server 140, and the viewer 150 are connected together through a network 130.

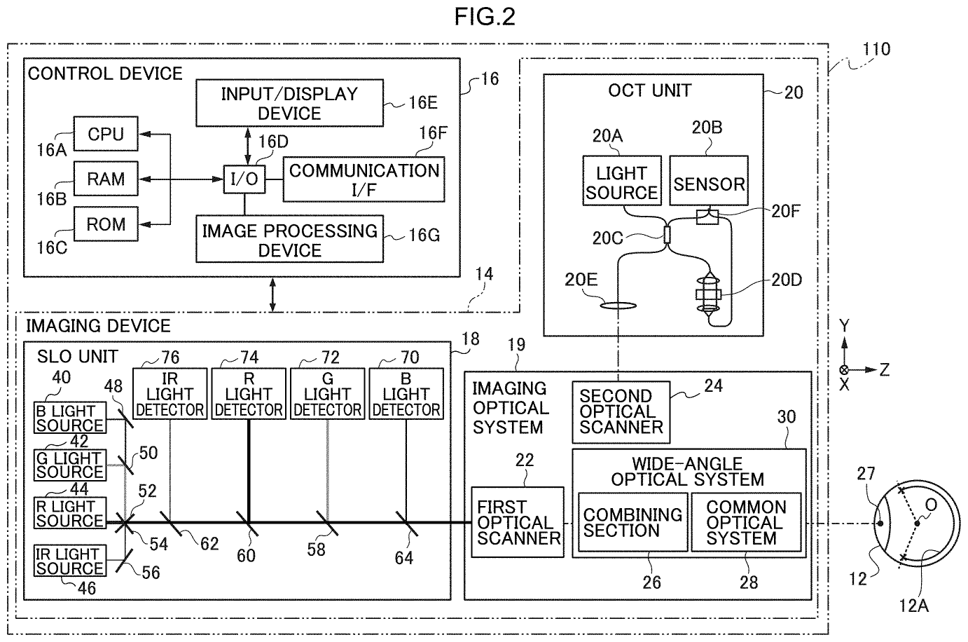

Next, explanation follows regarding a configuration of the ophthalmic device 110, with reference to FIG. 2.

For ease of explanation, scanning laser ophthalmoscope is abbreviated to SLO. Optical coherence tomography is also abbreviated to OCT.

With the ophthalmic device 110 installed on a horizontal plane and a horizontal direction taken as an X direction, a direction perpendicular to the horizontal plane is denoted a Y direction, and a direction connecting the center of the pupil at the anterior eye portion of the examined eye 12 and the center of the eyeball is denoted a Z direction. The X direction, the Y direction, and the Z direction are thus mutually perpendicular directions.

The ophthalmic device 110 includes an imaging device 14 and a control device 16. The imaging device 14 is provided with an SLO unit 18, an OCT unit 20, and an imaging optical system 19, and acquires a fundus image of the fundus of the examined eye 12. Two-dimensional fundus images that have been acquired by the SLO unit 18 are referred to hereafter as SLO images. Tomographic images, face-on images (en-face images) and the like of the retina created based on OCT data acquired by the OCT unit 20 are referred to hereafter as OCT images.

The control device 16 includes a computer provided with a Central Processing Unit (CPU) 16A, Random Access Memory (RAM) 16B, Read-Only Memory (ROM) 16C, and an input/output (I/O) port 16D.

The control device 16 is provided with an input/display device 16E connected to the CPU 16A through the I/O port 16D. The input/display device 16E includes a graphical user interface to display images of the examined eye 12 and to receive various instructions from a user. An example of the graphical user interface is a touch panel display.

The control device 16 is also provided with an image processing device 16G connected to the I/O port 16D. The image processing device 16G generates images of the examined eye 12 based on data acquired by the imaging device 14. The control device 16 is also provided with a communication interface (I/F) 16F connected to the I/O port 16D. The ophthalmic device 110 is connected to the eye axial length measurement device 120, the server 140, and the viewer 150 through the communication interface (I/F) 16F and the network 130.

Although the control device 16 of the ophthalmic device 110 is provided with the input/display device 16E as illustrated in FIG. 2, the technology disclosed herein is not limited thereto. For example, a configuration may adopted in which the control device 16 of the ophthalmic device 110 is not provided with the input/display device 16E, and instead a separate input/display device is provided that is physically independent of the ophthalmic device 110. In such cases, the display device is provided with an image processing processor unit that operates under the control of the CPU 16A in the control device 16. Such an image processing processor unit may display SLO images and the like based on an image signal output as an instruction by the CPU 16A.

The imaging device 14 operates under the control of the CPU 16A of the control device 16. The imaging device 14 includes the SLO unit 18, the imaging optical system 19, and the OCT unit 20. The imaging optical system 19 includes a first optical scanner 22, a second optical scanner 24, and a wide-angle optical system 30.

The first optical scanner 22 scans light emitted from the SLO unit 18 two dimensionally in the X direction and the Y direction. The second optical scanner 24 scans light emitted from the OCT unit 20 two dimensionally in the X direction and the Y direction. As long as the first optical scanner 22 and the second optical scanner 24 are optical elements capable of deflecting light beams, they may be configured by any out of, for example, polygon mirrors, mirror galvanometers, or the like. A combination thereof may also be employed.

The wide-angle optical system 30 includes an objective optical system (not illustrated in FIG. 2) provided with a common optical system 28, and a combining section 26 that combines light from the SLO unit 18 with light from the OCT unit 20.

The objective optical system of the common optical system 28 may be a reflection optical system employing a concave mirror such as an elliptical mirror, a refraction optical system employing a wide-angle lens, or may be a reflection-refraction optical system employing a combination of a concave mirror and a lens. Employing a wide-angle optical system that utilizes an elliptical mirror, wide-angle lens, or the like enables imaging to be performed not only of a central portion of the fundus where the optic nerve head and macular are present, but also of the retina at a peripheral portion of the fundus where an equatorial portion of the eyeball and vortex veins are present.

For a system including an elliptical mirror, a configuration may be adopted that utilizes an elliptical mirror system as disclosed in International Publication (WO) Nos. 2016/103484 or 2016/103489. The disclosures of WO Nos. 2016/103484 and 2016/103489 are incorporated in their entirety by reference herein.

Observation of the fundus over a wide field of view (FOV) 12A is implemented by employing the wide-angle optical system 30. The FOV 12A refers to a range capable of being imaged by the imaging device 14. The FOV 12A may be expressed as a viewing angle. In the present exemplary embodiment the viewing angle may be defined in terms of an internal illumination angle and an external illumination angle. The external illumination angle is the angle of illumination by a light beam shone from the ophthalmic device 110 toward the examined eye 12, and is an angle of illumination defined with respect to a pupil 27. The internal illumination angle is the angle of illumination of a light beam shone onto the fundus, and is an angle of illumination defined with respect to an eyeball center O. A correspondence relationship exists between the external illumination angle and the internal illumination angle. For example, an external illumination angle of 120° is equivalent to an internal illumination angle of approximately 160°. The internal illumination angle in the present exemplary embodiment is 200°.

An angle of 200° for the internal illumination angle is an example of a “specific value” of technology disclosed herein.

SLO fundus images obtained by imaging at an imaging angle having an internal illumination angle of 160° or greater are referred to as UWF-SLO fundus images. UWF is an abbreviation of ultra-wide field. Obviously an SLO image that is not UWF can be acquired by imaging the fundus at an imaging angle that is an internal illumination angle of less than 160°.

An SLO system is realized by the control device 16, the SLO unit 18, and the imaging optical system 19 as illustrated in FIG. 2. The SLO system is provided with the wide-angle optical system 30, enabling fundus imaging over the wide FOV 12A.

The SLO unit 18 is provided with plural light sources such as, for example, a blue (B) light source 40, a green (G) light source 42, a red (R) light source 44, an infrared (for example near infrared) (IR) light source 46, and optical systems 48, 50, 52, 54, 56 to guide the light from the light sources 40, 42, 44, 46 onto a single optical path using reflection or transmission. The optical systems 48, 50, 56 are configured by mirrors, and the optical systems 52, 54 are configured by beam splitters. B light is reflected by the optical system 48, is transmitted through the optical system 50, and is reflected by the optical system 54. G light is reflected by the optical systems 50, 54, R light is transmitted through the optical systems 52, 54, and IR light is reflected by the optical systems 56, 52. The respective lights are thereby guided onto a single optical path.

The SLO unit 18 is configured so as to be capable of switching between the light source or the combination of light sources employed for emitting laser light of different wavelengths, such as a mode in which G light, R light and B light are emitted, a mode in which infrared light is emitted, etc. Although the example in FIG. 2 includes four light sources, i.e. the B light source 40, the G light source 42, the R light source 44, and the IR light source 46, the technology disclosed herein is not limited thereto. For example, the SLO unit 18 may, furthermore, also include a white light source, in a configuration in which light is emitted in various modes, such as a mode in which white light is emitted alone.

Light introduced to the imaging optical system 19 from the SLO unit 18 is scanned in the X direction and the Y direction by the first optical scanner 22. The scanning light passes through the wide-angle optical system 30 and the pupil 27 and is shone onto the posterior eye portion of the examined eye 12. Reflected light that has been reflected by the fundus passes through the wide-angle optical system 30 and the first optical scanner 22 and is introduced into the SLO unit 18.

The SLO unit 18 is provided with a beam splitter 64 that, from out of the light coming from the posterior eye portion (e.g. fundus) of the examined eye 12, reflects the B light therein and transmits light other than B light therein, and a beam splitter 58 that, from out of the light transmitted by the beam splitter 64, reflects the G light therein and transmits light other than G light therein. The SLO unit 18 is further provided with a beam splitter 60 that, from out of the light transmitted through the beam splitter 58, reflects R light therein and transmits light other than R light therein. The SLO unit 18 is further provided with a beam splitter 62 that reflects IR light from out of the light transmitted through the beam splitter 60.

The SLO unit 18 is provided with plural light detectors corresponding to the plural light sources. The SLO unit 18 includes a B light detector 70 for detecting B light reflected by the beam splitter 64, and a G light detector 72 for detecting G light reflected by the beam splitter 58. The SLO unit 18 also includes an R light detector 74 for detecting R light reflected by the beam splitter 60 and an IR light detector 76 for detecting IR light reflected by the beam splitter 62.

Light that has passed through the wide-angle optical system 30 and the first optical scanner 22 and been introduced into the SLO unit 18 (i.e. reflected light that has been reflected by the fundus) is reflected by the beam splitter 64 and photo-detected by the B light detector 70 when B light, and is transmitted through the beam splitter 64 and reflected by the beam splitter 58 and photo-detected by the G light detector 72 when G light. When R light, the incident light is transmitted through the beam splitters 64, 58, reflected by the beam splitter 60, and photo-detected by the R light detector 74. When IR light, the incident light is transmitted through the beam splitters 64, 58, 60, reflected by the beam splitter 62, and photo-detected by the IR light detector 76. The image processing device 16G that operates under the control of the CPU 16A employs signals detected by the B light detector 70, the G light detector 72, the R light detector 74, and the IR light detector 76 to generate UWF-SLO images.

The UWF-SLO image (sometimes referred to as a UWF fundus image or an original fundus image as described later) encompasses a UWF-SLO image (green fundus image) obtained by imaging the fundus in green, and a UWF-SLO image (red fundus image) obtained by imaging the fundus in red. The UWF-SLO image further encompasses a UWF-SLO image (blue fundus image) obtained by imaging the fundus in blue, and a UWF-SLO image (IR fundus image) obtained by imaging the fundus in IR.

The control device 16 also controls the light sources 40, 42, 44 so as to emit light at the same time. A green fundus image, a red fundus image, and a blue fundus image are obtained with mutually corresponding positions by imaging the fundus of the examined eye 12 at the same time with the B light, G light, and R light. An RGB color fundus image is obtained from the green fundus image, the red fundus image, and the blue fundus image. The control device 16 obtains a green fundus image and a red fundus image with mutually corresponding positions by controlling the light sources 42, 44 so as to emit light at the same time and by imaging the fundus of the examined eye 12 at the same time with the G light and R light. A RG color fundus image is obtained from the green fundus image and the red fundus image.

Specific examples of the UWF-SLO image include a blue fundus image, a green fundus image, a red fundus image, an IR fundus image, an RGB color fundus image, and an RG color fundus image. The image data for the respective UWF-SLO images are transmitted from the ophthalmic device 110 to the server 140 through the communication interface (I/F) 16F, together with patient information input through the input/display device 16E. The image data of the respective UWF-SLO images and the patient information are stored associated with each other in a storage device 254. The patient information includes, for example, patient ID, name, age, visual acuity, right eye/left eye discriminator, and the like. The patient information is input by an operator through the input/display device 16E.

An OCT system is realized by the control device 16, the OCT unit 20, and the imaging optical system 19 illustrated in FIG. 2. The OCT system is provided with the wide-angle optical system 30. This enables fundus imaging to be performed over the wide FOV 12A similarly to when imaging the SLO fundus images as described above. The OCT unit 20 includes a light source 20A, a sensor (detector) 20B, a first light coupler 20C, a reference optical system 20D, a collimator lens 20E, and a second light coupler 20F.

Light emitted from the light source 20A is split by the first light coupler 20C. After one part of the split light has been collimated by the collimator lens 20E into parallel light to serve as measurement light, the parallel light is introduced into the imaging optical system 19. The measurement light is scanned in the X direction and the Y direction by the second optical scanner 24. The scanning light is shone onto the fundus through the wide-angle optical system 30 and the pupil 27. Measurement light that has been reflected by the fundus passes through the wide-angle optical system 30 and the second optical scanner 24 so as to be introduced into the OCT unit 20. The measurement light then passes through the collimator lens 20E and the first light coupler 20C before being incident to the second light coupler 20F.

The other part of the light emitted from the light source 20A and split by the first light coupler 20C is introduced into the reference optical system 20D as reference light, and is made incident to the second light coupler 20F through the reference optical system 20D.

The respective lights that are incident to the second light coupler 20F, namely the measurement light reflected by the fundus and the reference light, interfere with each other in the second light coupler 20F so as to generate interference light. The interference light is photo-detected by the sensor 20B. The image processing device 16G operating under the control of the CPU 16A generates OCT images, such as tomographic images and en-face images, based on OCT data detected by the sensor 20B.

OCT fundus images obtained by imaging at an imaging angle having an internal illumination angle of 160° or greater are referred to as UWF-OCT images. Obviously OCT data can be acquired at an imaging angle having an internal illumination angle of less than 160°.

The image data of the UWF-OCT images is transmitted, together with the patient information, from the ophthalmic device 110 to the server 140 though the communication interface (I/F) 16F. The image data of the UWF-OCT images and the patient information are stored associated with each other in the storage device 254.

Note that although in the present exemplary embodiment an example is given in which the light source 20A is a swept-source OCT (SS-OCT), the light source 20A may be configured from various types of OCT system, such as a spectral-domain OCT (SD-OCT) or a time-domain OCT (TD-OCT) system.

Next, explanation follows regarding the eye axial length measurement device 120. The eye axial length measurement device 120 has two modes, i.e. a first mode and a second mode, for measuring eye axial length, this being the length of an examined eye 12 in an eye axial direction. In the first mode, light from a non-illustrated light source is guided into the examined eye 12. Interference light between light reflected from the fundus and light reflected from the cornea is photo-detected, and the eye axial length is measured based on an interference signal representing the photo-detected interference light. The second mode is a mode to measure the eye axial length by employing non-illustrated ultrasound waves.

The eye axial length measurement device 120 transmits the eye axial length as measured using either the first mode or the second mode to the server 140. The eye axial length may be measured using both the first mode and the second mode, and in such cases, an average of the eye axial lengths as measured using the two modes is transmitted to the server 140 as the eye axial length. The server 140 stores the eye axial length of the patients in association with patient ID.

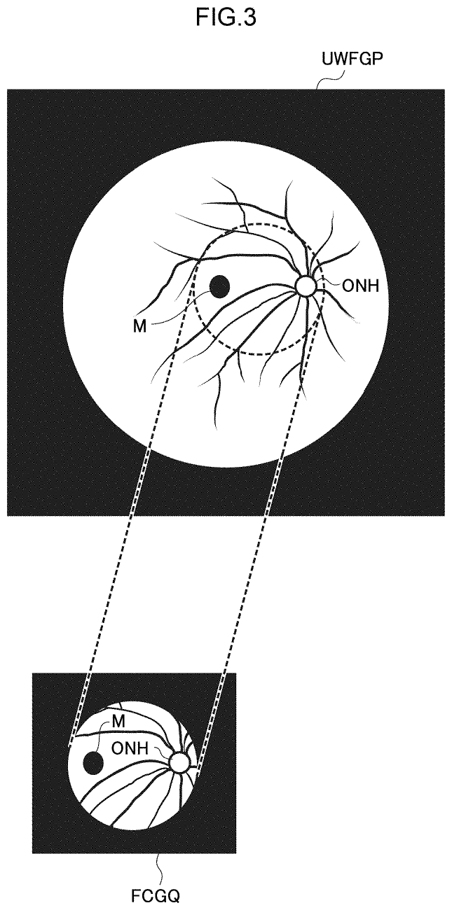

FIG. 3 illustrates an RG color fundus image UWFGP, and a fundus image FCGQ (fundus camera image) obtained by imaging a fundus of an examined eye 12 using a non-illustrated fundus camera. The RG color fundus image UWFGP is an image obtained by imaging the fundus at an imaging angle having an external illumination angle of 100°. The fundus image FCGQ (fundus camera image) is an image obtained by imaging the fundus at an imaging angle having an external illumination angle of 35°. Thus, as illustrated in FIG. 3, the fundus image FCGQ (fundus camera image) is a fundus image of an area that is part of the fundus region corresponding to the RG color fundus image UWFGP.

A UWF-SLO image such as the RG color fundus image UWFGP illustrated in FIG. 3 is an image having an area at the periphery of the image that is black due to light reflected from the fundus not arriving. Thus the UWF-SLO image includes a black area where light reflected from the fundus does not arrive (a background area, described later), and a part area of the fundus where light reflected from the fundus does arrive (a foreground area, described later). There are large differences between pixel values of each area at a boundary between the black area where light reflected from the fundus does not arrive and the part area of the fundus where light reflected from the fundus does arrive, and so the boundary is clear.

In contrast thereto, in the fundus image FCGQ (fundus camera image), a part area of the fundus where light reflected from the fundus does arrive (a foreground area, described later) is surrounded by flare, and a boundary between the foreground area needed for diagnosis and the background area not needed for diagnosis is not clear. Thus hitherto a specific mask image has been overlaid on the periphery of the foreground area, or pixel values of a specific area of the periphery of the foreground area have been overwritten with black pixel values. This makes a clear boundary between the black area where light reflected from the fundus does not arrive and the part area of the fundus where light reflected from the fundus does arrive.

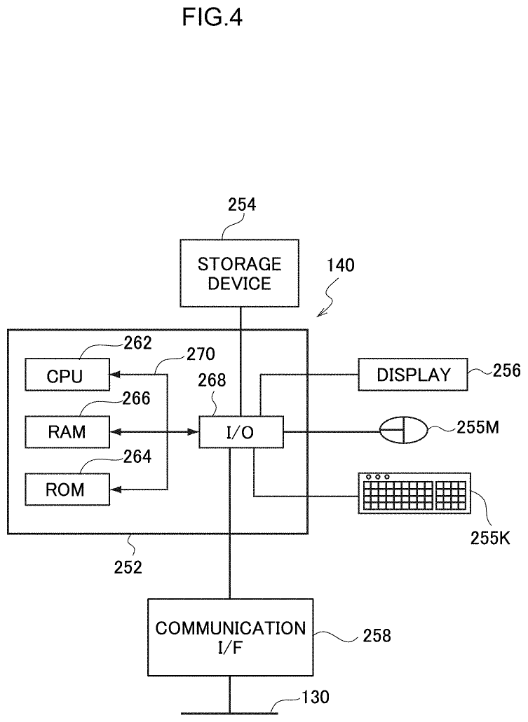

Explanation follows regarding a configuration of an electrical system of the server 140, with reference to FIG. 4. As illustrated in FIG. 4, the server 140 is provided with a computer body 252. The computer body 252 includes a CPU 262, RAM 266, ROM 264, and an input/output (I/O) port 268 connected together by a bus 270. The storage device 254, a display 256, a mouse 255M, a keyboard 255K, and a communication interface (I/F) 258 are connected to the input/output (I/O) port 268. The storage device 254 is, for example, configured by non-volatile memory. The input/output (I/O) port 268 is connected to the network 130 through the communication interface (I/F) 258. The server 140 is thus capable of communicating with the ophthalmic device 110 and the viewer 150. The storage device 254 is stored with an image processing program, described later. Note that the image processing program may be stored in the ROM 264.

The image processing program is an example of a “program” of technology disclosed herein. The storage device 254 and the ROM 264 are examples of “memory” and “computer readable storage medium” of technology disclosed herein. The CPU 262 is an example of a “processor” of technology disclosed herein.

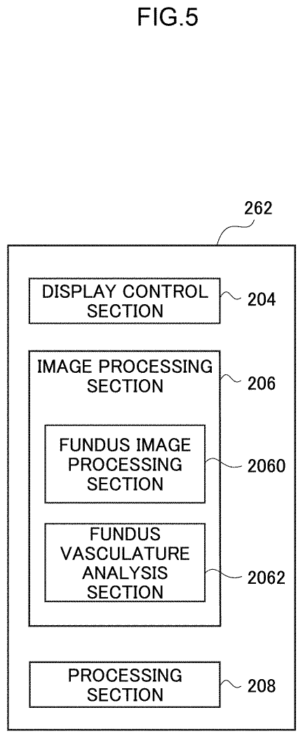

A processing section 208, described later, of the server 140 (see also FIG. 5) stores various data received from the ophthalmic device 110 in the storage device 254. More specifically, the processing section 208 stores respective image data of the UWF-SLO images and image data of the UWF-OCT images in the storage device 254 associated with the patient information (such as the patient ID as described above). Moreover, in cases in which there is a pathological change in the examined eye of the patient and cases in which surgery has been performed on a pathological lesion, pathology information is input through the input/display device 16E of the ophthalmic device 110 and transmitted to the server 140. The pathology information is stored in the storage device 254 associated with the patient information. The pathology information includes information about the position of the pathological lesion, name of the pathological change, and name of the surgery and date/time of surgery etc. when surgery was performed on the pathological lesion.

The viewer 150 is provided with a computer equipped with a CPU, RAM, ROM and the like, and a display. The image processing program is installed in the ROM, and based on an instruction from a user, the computer controls the display so as to display the medical information such as fundus images acquired from the server 140.

Next, description follows regarding various functions implemented by the CPU 262 of the server 140 executing the image processing program, with reference to FIG. 5. The image processing program includes a display control function, an image processing function (fundus image processing function, fundus vasculature analysis function), and a processing function. By the CPU 262 executing the image processing program including these various functions, the CPU 262 functions as a display control section 204, an image processing section 206 (fundus image processing section 2060, fundus vasculature analysis section 2062), and the processing section 208, as illustrated in FIG. 5.

The fundus image processing section 2060 is an example of an “acquisition section” and a “generation section” of technology disclosed herein.

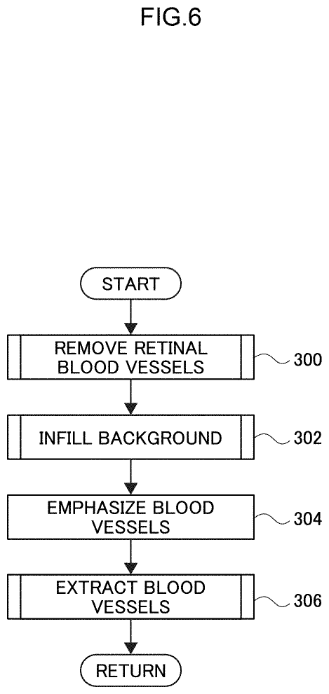

Detailed explanation now follows regarding image processing by the server 140, with reference to FIG. 6. The image processing and an image processing method illustrated by the flowchart in FIG. 6 is implemented by the CPU 262 of the server 140 executing the image processing program.

The image processing program starts when image data of a fundus image acquired by imaging the fundus of the examined eye 12 using the ophthalmic device 110 has been transmitted from the ophthalmic device 110 and received by the server 140.

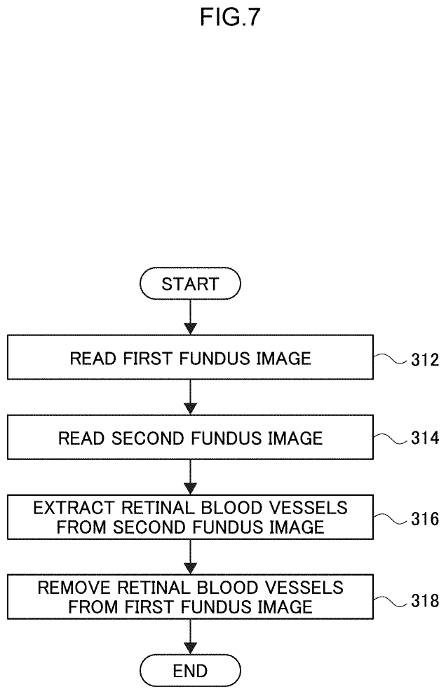

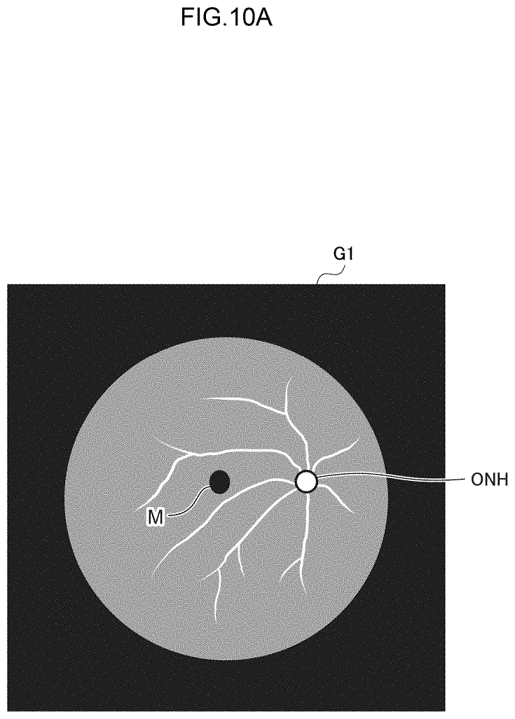

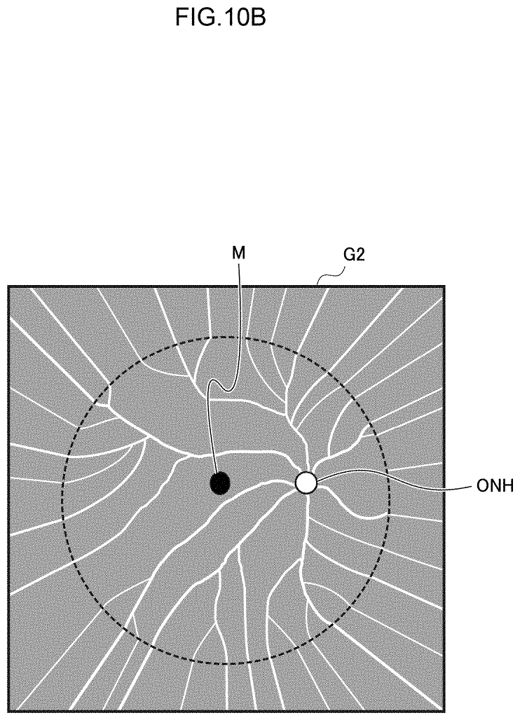

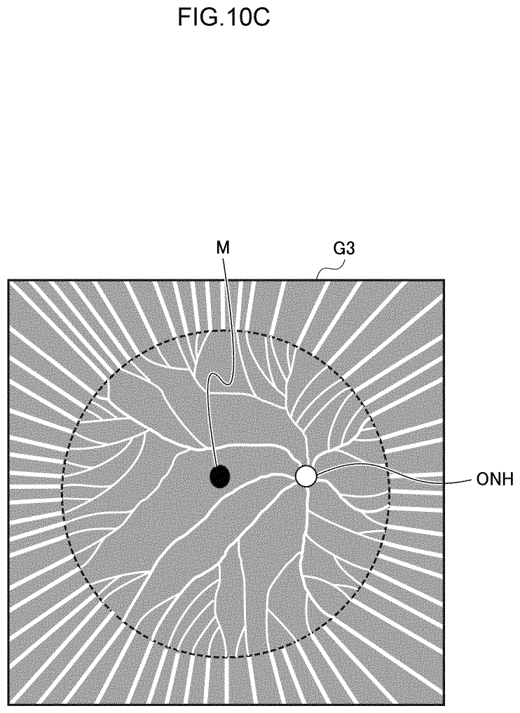

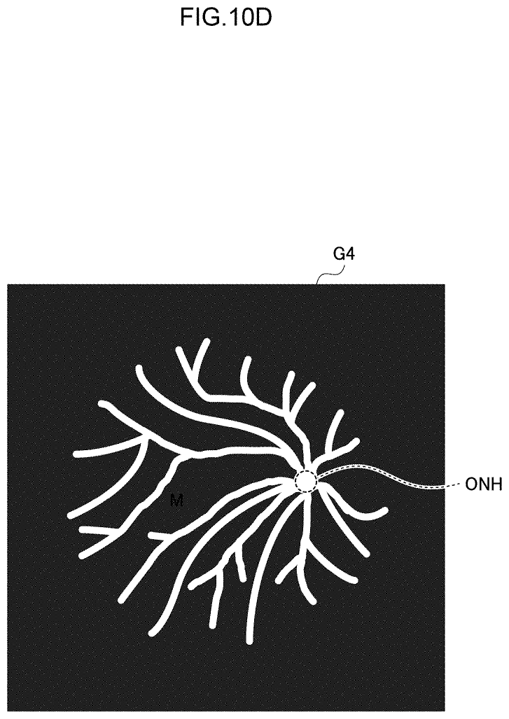

When the image processing program has started, at step 300 the fundus image processing section 2060 acquires the fundus image, and executes retinal blood vessel removal processing to remove the retinal blood vessels from the acquired fundus image, described in detail later (see FIG. 7). A choroidal vascular image G1 illustrated in FIG. 10A is generated by the processing of step 300.

The choroidal vascular image G1 is an example of a “first fundus image” of technology disclosed herein.

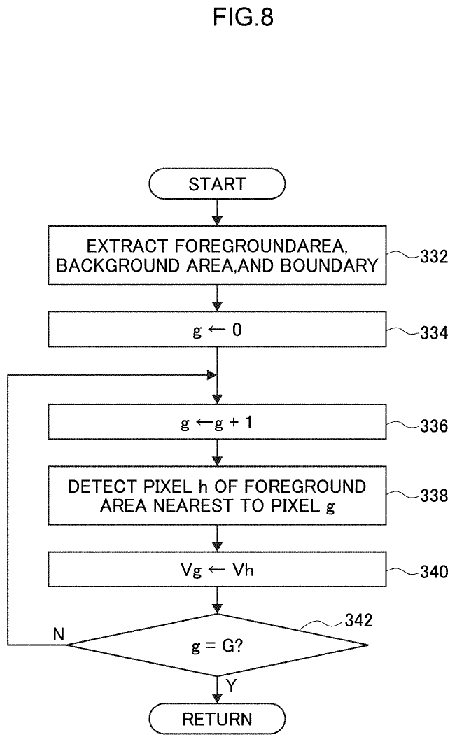

At step 302 the fundus image processing section 2060 executes background infill processing to infill each of the pixels of a background area with pixel values of pixels of the image of a foreground area having the shortest distance to the respective pixel, described in detail later (see FIG. 8). A background processing complete image G2 illustrated in FIG. 10B is generated by the background infill processing of step 302. Note that in FIG. 10B a range within the circular intermittent line is the fundus region.

The background infill processing of step 302 is an example of “background processing” of technology disclosed herein, the background processing complete image G2 is an example of a “second fundus image” of technology disclosed herein.

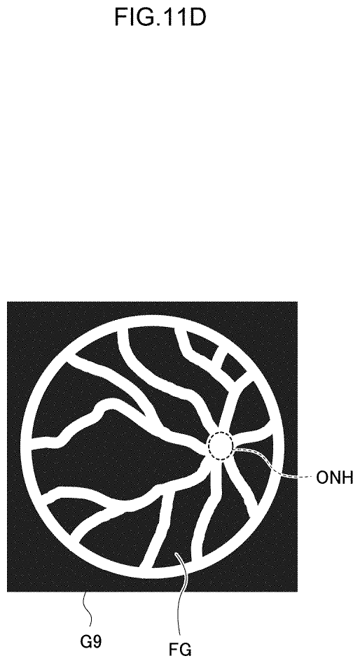

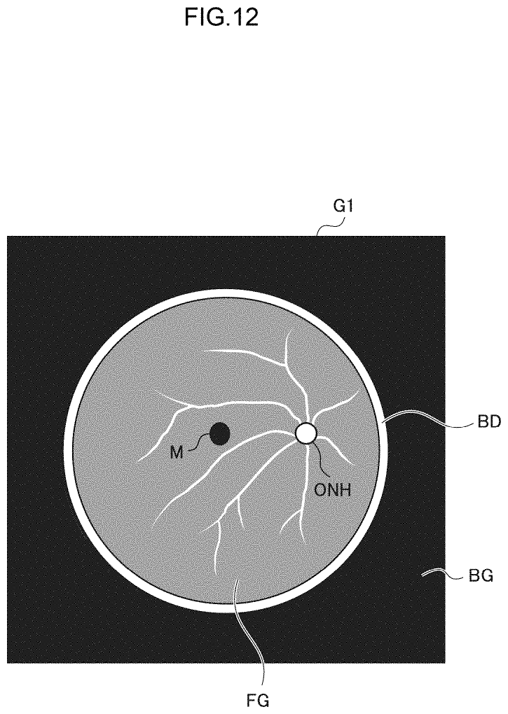

Explanation follows regarding the foreground area and the background area. As illustrated in FIG. 12, a foreground area FG in the choroidal vascular image G1 is determined by an arrival area of light from the fundus region of the examined eye 12, and is a pixel area of brightness values based on the intensity of light reflected from the examined eye 12 (in other words, an area depicting the fundus, namely an area of a fundus image of the examined eye 12). In contrast thereto, a background area BG is an area outside the fundus region of the examined eye 12, is a single color area, and is an image not based on light reflected from the examined eye 12. More specifically, the background area BG is an area not depicting the fundus, namely is a part outside the fundus region of the examined eye 12, and more precisely is a part including an area corresponding to pixels of the detectors 70, 72, 74, 76 where light reflected from the examined eye 12 does not arrive, a mask area, and parts of artefacts occurring due to vignetting, background reflections of the device, eyelid of the examined eye, and the like. Moreover, in cases in which the ophthalmic device 110 has a function to image an anterior eye portion area (cornea, iris, reticular formation, lens body, or the like), a specific area is the anterior eye portion area, and an anterior eye portion image of the examined eye is configured by the foreground area and the background area. Blood vessels appear in the reticular formation, and the technology disclosed herein enables the extraction of blood vessels of the reticular formation from the anterior eye portion image.

The fundus region of the examined eye 12 is an example of a “specific area of the examined eye” of technology disclosed herein.

At step 304 the fundus vasculature analysis section 2062 executes blood vessel emphasis processing on the background processing complete image G2 so as to generate a blood vessel emphasis image G3 illustrated in FIG. 10C. Contrast limited adaptive histogram equalization (CLAHE) may be employed as the blood vessel emphasis processing. Contrast limited adaptive histogram equalization (CLAHE) is a method of subdividing image data into plural areas, executing local histogram equalization on each of the subdivided areas, and adjusting the contrast by performing interpolation processing such as bilinear interpolation at boundaries between the respective areas. The blood vessel emphasis processing is not limited to contrast limited adaptive histogram equalization (CLAHE), and another method may be employed therefor. For example, unsharp mask processing (frequency processing), deconvolution processing, histogram equalization processing, haze removal processing, color correction processing, de-noise processing, or the like, or a combination processing thereof, may be employed.

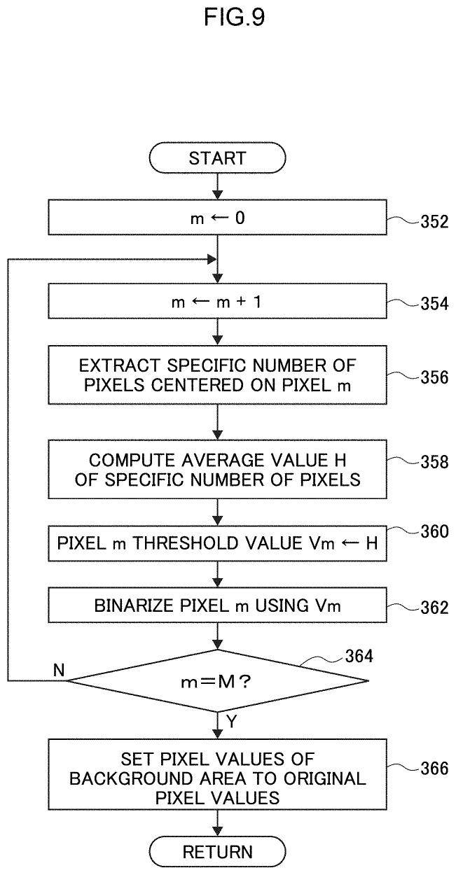

At step 306, the fundus image processing section 2060 generates a blood vessel extraction image (binarized image) G4 illustrated in FIG. 10D by extracting (specifically binarizing) blood vessels in the blood vessel emphasis image G3, described in detail later (see FIG. 9). In such a binarized image, the pixels of the blood vessel area are white, and the pixels of other areas are black, and there is no discrimination between the fundus region and the background area. Thus a fundus region is detected in advance by image processing and stored. Then based on this stored fundus region, a line segment is displayed overlaid on the boundary of the fundus region of the generated blood vessel extraction image (binarized image) G4. By overlaying the line segment indicating this boundary a user is able to discriminate between the fundus region and the background area.

The blood vessel extraction image G4 is an example of a “third fundus image” of technology disclosed herein.

Next with reference to FIG. 7, explanation follows regarding the retinal blood vessel removal processing of step 300 of FIG. 6.

At step 312 the fundus image processing section 2060 reads (acquires) image data of a first fundus image (red fundus image) from the image data of fundus images received from the ophthalmic device 110. At step 314 the fundus image processing section 2060 reads (acquires) image data of a second fundus image (green fundus image) from the image data of fundus images received from the ophthalmic device 110.

Explanation follows regarding information contained in the first fundus image (red fundus image) and the second fundus image (green fundus image).

The structure of an eye is one in which a vitreous body is covered by plural layers of differing structure. The plural layers include, from the vitreous body at the extreme inside to the outside, the retina, the choroid, and the sclera. R light passes through the retina and reaches the choroid. The first fundus image (red fundus image) therefore includes information relating to blood vessels present within the retina (retinal blood vessels) and information relating to blood vessels present within the choroid (choroidal blood vessels). In contrast thereto, G light only reaches as far as the retina. The second fundus image (green fundus image) accordingly only includes information relating to the blood vessels present within the retina (retinal blood vessels).

At step 316 the fundus image processing section 2060 performs black hat filter processing on the second fundus image (green fundus image) so as to extract the retinal blood vessels visible as thin black lines in the second fundus image (green fundus image). The black hat filter processing is filter processing to extract fine lines.

The black hat filter processing is processing to find a difference between image data of the second fundus image (green fundus image), and image data obtained by closing processing in which dilation processing is performed N times on the source image data followed by performing erosion processing N times (wherein N is an integer of 1 or more). In a fundus image the retinal blood vessels are imaged blacker than the periphery of the blood vessels because illumination light (not only G light but also R light or IR light) is absorbed by the retinal blood vessels. Thus the retinal blood vessels can be extracted by performing black hat filter processing on the fundus image.

At step 318 the fundus image processing section 2060 removes the retinal blood vessels extracted at step 316 from the first fundus image (red fundus image) by performing in-painting processing thereon. More specifically, the retinal blood vessels are made to no longer stand out in the first fundus image (red fundus image). Even more precisely, the fundus image processing section 2060 identifies, in the first fundus image (red fundus image), each of the positions of the retinal blood vessels extracted from the second fundus image (green fundus image). The fundus image processing section 2060 then performs processing such that a difference between pixel values of pixels in the first fundus image (red fundus image) at the identified positions, and an average value of pixels at the periphery of these pixels, is within a specific range (for example, zero). The method of removing retinal blood vessels is not limited to the example described above, and general in-painting processing may be employed therefor.

The retinal blood vessels do not stand out in the first fundus image (red fundus image) where both the retinal blood vessels and the choroidal blood vessels are present, and the fundus image processing section 2060 is accordingly able to make the choroidal blood vessels stand out comparatively more in the first fundus image (red fundus image) as a result of the above. As illustrated in FIG. 10A, the choroidal vascular image G1 is accordingly obtained in which only the choroidal blood vessels are visible as fundus blood vessels. Note that in FIG. 10A, the white line shapes are the choroidal blood vessels, and the white circular portion corresponds to the optic nerve head ONH, and the black circular portion corresponds to the macular M.

When the processing of step 318 has finished the retinal blood vessel removal processing of step 300 of FIG. 5 is ended, and the image processing transitions to step 302 of FIG. 6.

Next, with reference to FIG. 8 explanation follows to the background infill processing of step 302 of FIG. 6.

At step 332, as illustrated in FIG. 12, the fundus image processing section 2060 extracts the foreground area FG, the background area BG, and a boundary BD between the foreground area FG and the background area BG in the choroidal vascular image G1.

More specifically, the fundus image processing section 2060 extracts as the background area BG parts where the pixel value is zero, extracts as the foreground area FG parts where the pixel value is non-zero, and extracts as the boundary BD boundary sections between the extracted background area BG and the extracted foreground area FG.

As described above, in the background area BG the light from the examined eye 12 does not arrive, resulting in a part where the pixel values are zero. However, sometimes areas such as artefacts due to vignetting, background reflections of the device, eyelid of the examined eye, and the like are recognized as background area. Moreover, there are also cases in which pixel values of pixels in the area of a detector where light reflected from the examined eye 12 does not enter are not zero due to the sensitivity of the detectors 70, 72, 74, 76. The fundus image processing section 2060 may accordingly extract as the background area BG parts having a pixel value greater than a specific value greater than zero.

However, areas where light from the examined eye 12 arrives in the detection fields of the detectors 70, 72, 74, 76 are predetermined as paths for light of the optical elements of the imaging optical system 19. The areas where light arrives from the examined eye 12 are the foreground area FG, the areas where light does not arrive from the examined eye 12 are the background area BG, and a boundary section between the background area BG and the foreground area FG may be extracted as the BD boundary as described above.

At step 334 the fundus image processing section 2060 sets a variable g to identify each of the pixels of the image in the background area BG to zero, and at step 336 the fundus image processing section 2060 increments variable g by one.

At step 338 the fundus image processing section 2060 detects a nearest pixel h of the foreground area FG having a closest distance to a pixel g of the background area BG image identified by variable g using relationships between the position of the pixel g and the positions of each of the pixels of the foreground area FG image. The fundus image processing section 2060 may, for example, calculate a distance between the position of the pixel g and the positions of each of the pixels of the foreground area FG image, and detect the pixel having the shortest distance as the pixel h. However, in the present exemplary embodiment the position of the pixel h is predetermined from the geometrical relationship between the position of the pixel g and the positions of each of the pixels of the foreground area FG image.

At step 340 the fundus image processing section 2060 sets a pixel value Vh different than the pixel value Vg for the pixel value Vg of the pixel g, for example, sets the pixel value Vh of the pixel h detected at step 338.

At step 342 the fundus image processing section 2060 determines whether or not a pixel value different than the pixel value has been set for the pixel values of all the pixels in the image of the background area BG by determining whether or not the variable g is equal to a total number G of the pixels in the image of the background area BG. The background infill processing returns to step 336 in cases in which the variable g is determined not to be equal to the total number G, and the fundus image processing section 2060 executes the above processing (from step 336 to step 342).

When determined that the variable g is equal to the total number G at step 342, this means that the pixel values of all of the pixels in the background area BG image have been converted into pixel values different than their respective pixel values, and so the background infill processing is ended.

The background processing complete image G2 illustrated in FIG. 10B is generated by the background infill processing of step 302 (steps 332 to 342 of FIG. 8).

Note that, as described in detail later, when calculating a threshold value for binarizing the pixel values of the pixels in the foreground area FG image, the fundus image processing section 2060 extracts a specific number of pixels centered on the respective pixel and employs an average of the pixel values for these extracted pixels. Thus it suffices to identify just the pixels that may be extracted to calculate the threshold value from out of the pixels of the background area BG image as the variable g. In such cases the total number G may be the total number of pixels that may be extracted when calculating the threshold value. In such cases the pixels identified by the variable g are the pixels surrounding the foreground area FG from out of the pixels of the background area BG image. Note that in such cases, moreover, a pixel may be identified by the variable g that is any one or more pixel from out of the pixels surrounding the foreground area FG.

In the background infill processing of step 302 (steps 332 to 342 of FIG. 8), pixel values of each of the pixels of the background area BG are sequentially converted to the pixel value of the nearest foreground area FG pixel having the closest distance to that respective pixel. The technology disclosed herein is not limited thereto.

### MODIFIED EXAMPLES OF BACKGROUND INFILL PROCESSING OF STEP 302

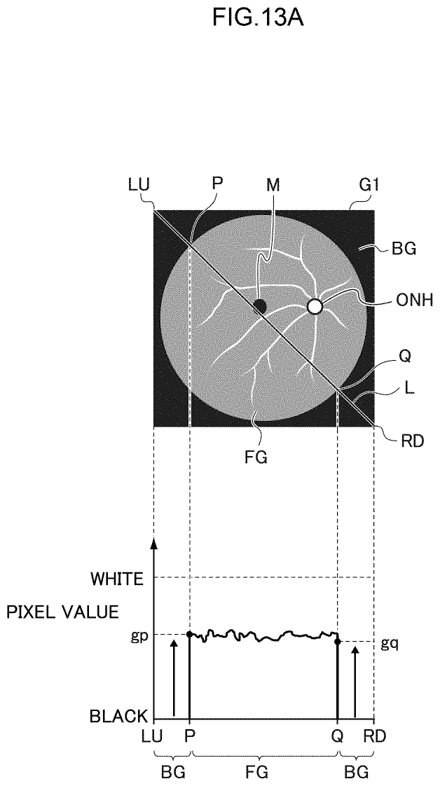

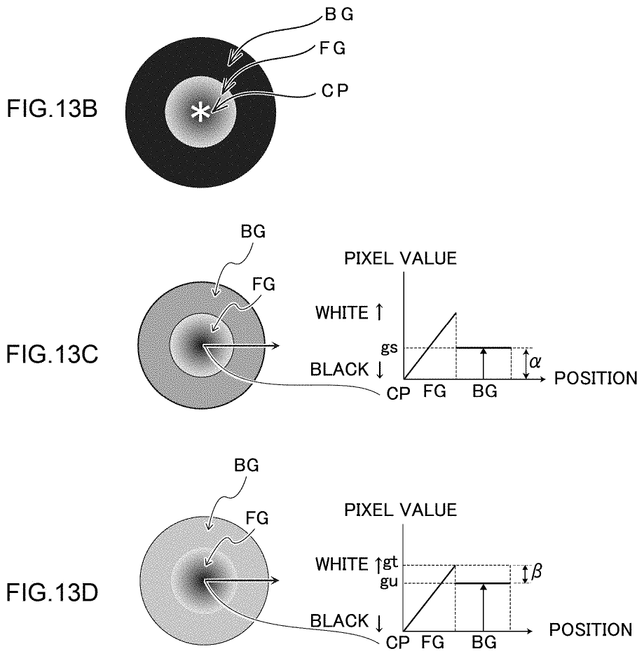

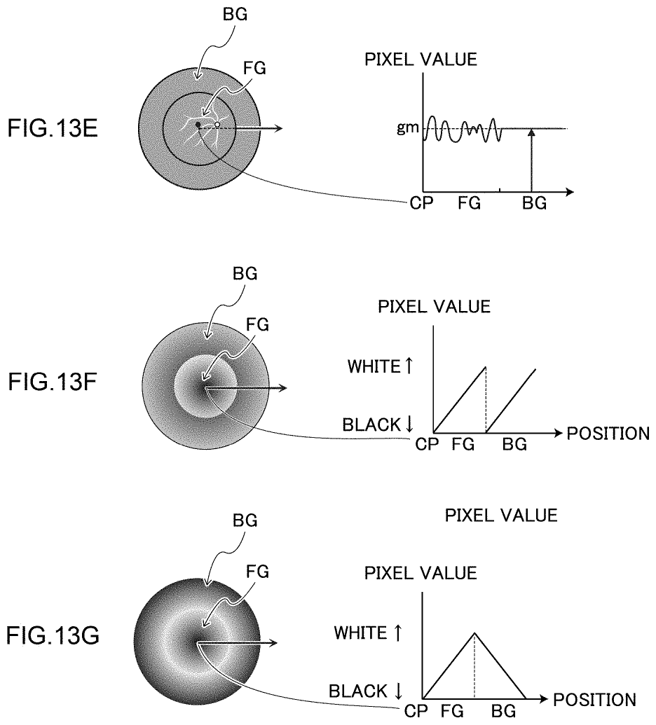

Next, description follows regarding modified examples of the background infill processing of step 302, with reference to FIG. 13A to FIG. 13G.

### Modified Example 1 of Background Infill Processing

As illustrated in FIG. 13A, for example, the fundus image processing section 2060 converts the pixel value of each of the pixels of the background area BG on a line L passing through a center of the choroidal vascular image G1 to the pixel value of the nearest foreground area FG pixel closest to each of the respective pixels. More specifically, the fundus image processing section 2060 extracts the line L from a pixel LU at one corner of the choroidal vascular image G1 and passing through the center thereof and passing through a pixel RD at another corner on the opposite side of the center. The fundus image processing section 2060 converts the pixel values of each of the pixels on the line L from the pixel LU at the one corner of the background area BG to the pixel of the background area BG adjacent to a nearest foreground area FG pixel P having the closest distance to the pixel LU, to a pixel value gp of the pixel P. The fundus image processing section 2060 converts the pixel values of each of the pixels on the line L from the pixel RD at the other corner of the background area BG to the pixel of the background area BG adjacent to a nearest foreground area FG pixel Q having the closest distance to the pixel RD, to a pixel value gq of the pixel Q. The fundus image processing section 2060 executes such pixel value conversion for all lines passing through the center of the choroidal vascular image G1.

### Modified Example 2 of Background Infill Processing

FIG. 13B schematically illustrates a choroidal vascular image G1 including a center position CP of the foreground area FG, the foreground area FG, and the background area BG surrounding the foreground area FG. More specifically the center position CP is indicated by the * mark. Light from the examined eye 12 arrives at each of the pixels of the foreground area FG image, and so they have pixel values according to the intensity of the arriving light, however, in FIG. 13B the pixel values are schematically illustrated as smoothly increasing in the foreground area FG from the center position CP toward the outside. The pixel values of the background area BG are illustrated as being zero.

In the Modified Example 2 of the background infill processing, as illustrated in FIG. 13C, the fundus image processing section 2060 converts the pixel values of each of the pixels of the background area BG image into a value gs (=0+α) that is greater than the respective pixel values by a specific value α.

### Modified Example 3 of Background Infill Processing

At step 302 the pixels of the background area BG image are converted to pixel values of the nearest foreground area FG pixel having the closest distance to the respective pixel. In contrast thereto, in the Modified Example 3 of the background infill processing, as illustrated in FIG. 13D, the fundus image processing section 2060 converts the pixels of the background area BG image to a value gu (=gt−α) smaller than a pixel value gt of the nearest foreground area FG pixel by a specific value β.

### Modified Example 4 of Background Infill Processing

In a Modified Example 4 of the background infill processing, as illustrated in FIG. 13E, the fundus image processing section 2060 converts the pixel value of each of the pixels of the background area BG to an average value gm of the pixel values for all the pixels of the foreground area FG.

### Modified Example 5 of Background Infill Processing

In a Modified Example 5 of the background infill processing, as illustrated in FIG. 13F, the fundus image processing section 2060 detects changes to the pixel values from the center pixel CP to an edge portion of the foreground area FG. The fundus image processing section 2060 then applies a change in pixel values in the background area BG that is similar to the change in the pixel values in the foreground area FG. Namely, pixel values from the center pixel CP to the edge portion of the foreground area FG are exchanged for the pixel values from the innermost perimeter of the background area BG to the outermost perimeter thereof.

In the example of the fundus image schematically illustrated in FIG. 13F, the pixel values smoothly increase in the foreground area FG from the center position CP toward the outside. In the Modified Example 5 the fundus image processing section 2060 converts each of the pixels of the background area BG image to values that are gradually greater the longer the distance is from the center CP of the foreground area FG.

### Modified Example 6 of Background Infill Processing

In the Modified Example 6 of the background infill processing, as illustrated in FIG. 13G, the fundus image processing section 2060 detects changes in pixel values from the center pixel CP to the edge portion of the foreground area FG. The fundus image processing section 2060 then applies changes in the background area BG that are the reverse of changes to the pixel values in the foreground area FG. Namely, pixel values from the edge portion of the foreground area FG to the center pixel CP are substituted for the pixel values from the innermost perimeter of the background area BG to the outermost perimeter thereof.

In the example of the fundus image schematically illustrated in FIG. 13G, the pixel values smoothly increase in the foreground area FG from the center position CP toward the outside. In the Modified Example 6 the fundus image processing section 2060 converts each of the pixels of the background area BG image to values that gradually decrease the longer the distance is from the center CP of the foreground area FG.

Moreover, the technology disclosed herein includes modifications to the content of the processing for Modified Example 1 to Modified Example 6 within a range not departing from the spirit of technology disclosed herein.

When the background infill processing has finished, the image processing proceeds to step 304 of FIG. 6, and at step 304 the blood vessel emphasis processing (for example CLAHE or the like) is executed as described above so as to generate the blood vessel emphasis image G3 illustrated in FIG. 10C.

The blood vessel emphasis image G3 is an example of an “image resulting from emphasizing blood vessels” of technology disclosed herein.

When the blood vessel emphasis processing of step 304 has finished the image processing proceeds to step 306 of FIG. 6.

Next, description follows regarding processing to extract blood vessels at step 306 of FIG. 6, with reference to FIG. 9.

At step 352 the fundus image processing section 2060 sets a variable m to identify each of the pixels of the foreground area FG image in the blood vessel emphasis image G3 to zero, and at step 354 the fundus image processing section 2060 increments the variable m by one.

At step 356 the fundus image processing section 2060 extracts a specific number of pixels centered on a pixel m of the foreground area FG identified by variable m. For example, the specific number of pixels extracted are four pixels adjacent above, below, to the left, and to the right of the pixel m, or a total of eight pixels adjacent thereto above, below, to the left, and to the right, and in diagonal directions. There is no limit to the adjacent eight pixels, and pixels in the vicinity may be extracted from a wider range.

At step 358 the fundus image processing section 2060 computes an average value H of the pixel values for the specific number of pixels extracted at step 356. At step 360 the fundus image processing section 2060 sets the average value H as a threshold value Vm for pixel m. At step 362 the fundus image processing section 2060 binarizes the pixel value of pixel m using the threshold value Vm (=H).

At step 364 the fundus image processing section 2060 determines whether or not the variable m is equal to the total pixel number M of the foreground area FG image. Not all of the pixels of the foreground area FG image have been binarized with the above threshold value unless the variable m is determined to be equal to the total pixel number M, and so the processing to extract the blood vessels returns to step 354, and the fundus image processing section 2060 executes the above processing (steps 354 to 364).

In cases in which the variable m is equal to the total pixel number M, the pixel values of all of the pixels in the foreground area FG image have been binarized, and so at step 366 the fundus image processing section 2060 sets the pixel values of the background area BG in the blood vessel emphasis image G3 to the same pixel value as their original respective pixel values. The blood vessel extraction image G4 illustrated in FIG. 10D is generated by the processing of step 366.

The pixel values of the background area BG in the blood vessel emphasis image G3 are an example of “second pixel values” of technology disclosed herein, and the original pixel values are an example of “first pixel values” and “third pixel values” of technology disclosed herein.

Note that in the technology disclosed herein there is no limitation to setting the pixel values of the background area BG in the blood vessel emphasis image G3 to the same pixel value as their original respective pixel values, and the pixel values of the background area BG in the blood vessel emphasis image G3 may be substituted with a pixel value that is different from the original pixel value.

After the blood vessel emphasis processing of step 304, the processing to extract blood vessels of step 306 is executed. The image subjected to the blood vessels extraction processing is accordingly the blood vessel emphasis image G3. However, the technology disclosed herein is no limited thereto. For example, after the background infill processing of step 302 the blood vessel emphasis processing of step 304 may be omitted, and the processing to extract blood vessels of step 306 may be executed. In such cases the image subjected to the blood vessels extraction processing is the background processing complete image G2.

At step 306 the fundus vasculature analysis section 2062 may further execute choroid analysis processing. As the choroid analysis processing, the fundus image processing section 2060 executes, for example, vortex vein position detection processing and processing to analyze asymmetry in running directions of the choroidal vasculature.

The choroid analysis processing is an example of “analysis processing” of technology disclosed herein.

The execution timing of the choroid analysis processing may, for example, be between the processing of step 364 and the processing of step 366, or may be after the processing of step 366.

In cases in which the choroid analysis processing is executed between the processing of step 364 and the processing of step 366, the image subjected to the choroid analysis processing is an image prior to setting the pixel values of the background area in the blood vessel emphasis image G3 to their original pixel values. Note that in cases in which the blood vessel emphasis processing of step 304 is omitted, the choroid analysis processing is executed on the background processing complete image G2.

In contrast thereto, in cases in which the choroid analysis processing is executed after the processing of step 366, the image subjected to the choroid analysis processing is the blood vessel extraction image G4. The subject image is an image in which only the choroidal blood vessels have been made visible.

The vortex veins are flow paths of blood flow flowing into the choroid, and there are from four to six vortex veins present toward the posterior pole of an equatorial portion of the eyeball. The vortex vein positions are detected based on the running direction of the choroidal blood vessels obtained by analyzing the subjected image.

The fundus image analysis section 2060 sets a movement direction of each of the choroidal blood vessels (blood vessel running direction) in the subjected image. More specifically, first the fundus image analysis section 2060 executes the following processing on each pixel in the subjected image. Namely, for each pixel the fundus image analysis section 2060 sets an area (cell) having the respective pixel at the center, and creates a histogram of brightness gradient direction at each of the pixels in the cells. Next, the fundus image analysis section 2060 takes the gradient direction having the lowest count in the histogram of the cells as the movement direction for the pixels in each of the cells. This gradient direction corresponds to the blood vessel running direction. Note that the reason for taking the gradient direction having the lowest count as the blood vessel running direction is as follows. The brightness gradient is small in the blood vessel running direction, whereas the brightness gradient is large in other directions (for example, there is a large difference in brightness between blood vessel and non-blood vessel tissue). Thus creating a histogram of brightness gradient for each of the pixels results in a small count in the blood vessel running direction. The blood vessel running direction at each of the pixels in the subjected image is set by the processing described above.

The fundus image processing section 2060 sets an initial position for M (natural number)×N (natural number) (=L) individual hypothetical particles. More specifically, the fundus image processing section 2060 sets a total of L initial positions at uniform spacings on the subjected image, with M positions in the vertical direction, and N positions in the horizontal direction.

The fundus image processing section 2060 estimates the position of the vortex veins. More specifically, the fundus image analysis section 2060 performs the following processing for each of the L positions. Namely, the fundus image analysis section 2060 acquires a blood vessel running direction at an initial position (one of the L positions), moves the hypothetical particle by a specific distance along the acquired blood vessel running direction, then re-acquires the blood vessel running direction at the moved-to position, before then moving the hypothetical particle by the specific distance along this acquired blood vessel running direction. This moving by the specific distance along the blood vessel running direction is repeated for a pre-set number of movement times. The above processing is executed for all the L positions. Points where a fixed number of the hypothetical particles or greater have congregated at this point in time are taken as the position of a vortex vein.

The positional information of the vortex veins (number of vortex veins, coordinates on the subjected image, etc.) are stored in the storage device 254. A method disclosed in Japanese Patent Application No. 2018-080273 and a method disclosed in WO No. PCT/JP2019/016652 may be employed as the method for detecting vortex veins. The disclosures of Patent Application No. 2018-080273 filed in Japan on Apr. 18, 2018 and WO No. PCT/JP2019/016652 filed internationally on Apr. 18, 2019 are incorporated in their entirety in the present specification by reference herein.

The processing section 208 stores at least the choroidal vascular image G1 and the blood vessel extraction image G4, the choroid analysis data (respective data indicating vortex vein positions and the asymmetry of the running direction of the choroidal blood vessels and the like), together with patient information (patient ID, name, age, visual acuity, right eye/left eye discriminator, eye axial length, etc.), in the storage device 254 (see FIG. 4). The processing section 208 may also save the RG color fundus image UWFGP (original fundus image) and an image of a processing process such as the background processing complete image G2 and the blood vessel emphasis image G3.

Note that in the present exemplary embodiment the processing section 208 stores the RG color fundus image UWFGP (original fundus image), the choroidal vascular image G1, the background processing complete image G2, the blood vessel emphasis image G3, the blood vessel extraction image G4, and choroid analysis data, together with patient information, in the storage device 254 (see FIG. 4).

Description follows regarding the display on the viewer 150 of the fundus image captured by the ophthalmic device 110 and a fundus camera and the fundus image from the image processing by the image processing program of FIG. 6.

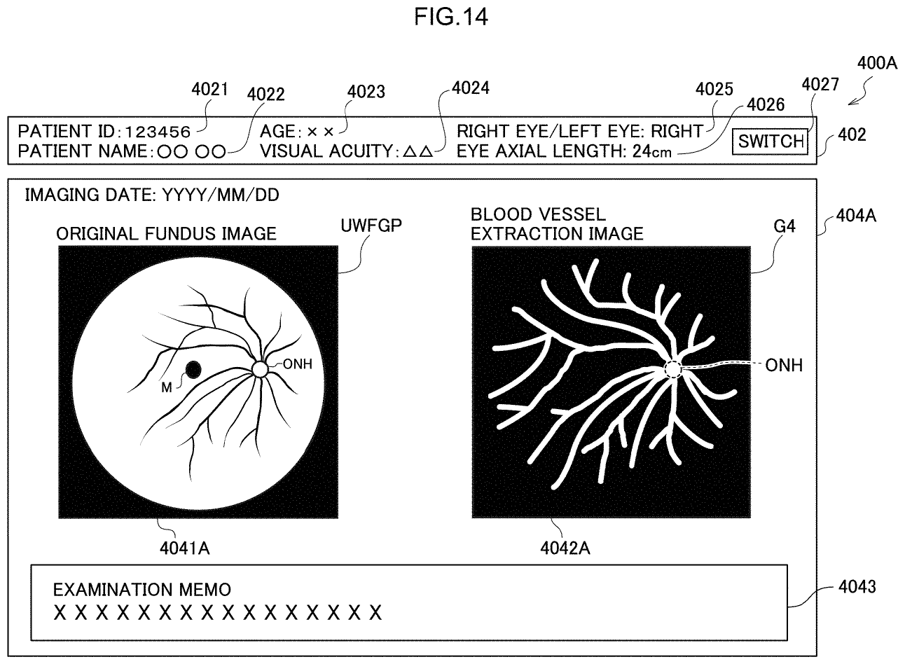

When an ophthalmologist is examining the examined eye 12 of a patient, the patient ID is input to the viewer 150. The viewer 150 input with the patient ID instructs the server 140 to transmit image data of each image (UWFGP, G1 to G4, etc.) together with patient information corresponding to the patient ID. The viewer 150 that has received the image data of each image (UWFGP, G1 to G4 etc.), together with the patient information, generates an examination screen 400A of the examined eye 12 of the patient, as illustrated in FIG. 14, and displays the examination screen 400A on the display of the viewer 150.

FIG. 14 illustrates the examination screen 400A of the viewer 150. The examination screen 400A as illustrated in FIG. 14 includes an information display area 402 and an image display area 404A.

The information display area 402 includes a patient ID display field 4021 and a patient name display field 4022. The information display area 402 also includes an age display field 4023 and a visual acuity display field 4024. The information display area 402 also includes a right eye/left eye information display field 4025 and an eye axial length display field 4026. The information display area 402 also includes a switch screen icon 4027. The viewer 150 displays information corresponding to each of the display fields (from 4021 to 4026) based on the patient information received.

The image display area 404A includes an original fundus image display field 4041A, a blood vessel extraction image display field 4042A, and a text display field 4043. The viewer 150 displays images (RG color fundus image UWFGP (original fundus image), blood vessel extraction image G4) corresponding to each display field (4041A, 4042A) based on the received image data. An imaging date (YYYY/MM/DD) when the images being displayed were acquired is also displayed in the image display area 404A.

An examination memo input by a user (ophthalmologist) is displayed in the text display field 4043. In addition, for example, text for analyzing the image being displayed such as “A choroidal vascular image is being displayed in the left side area. An image of extracted choroidal blood vessels is being displayed in the right side area”, may also be displayed.

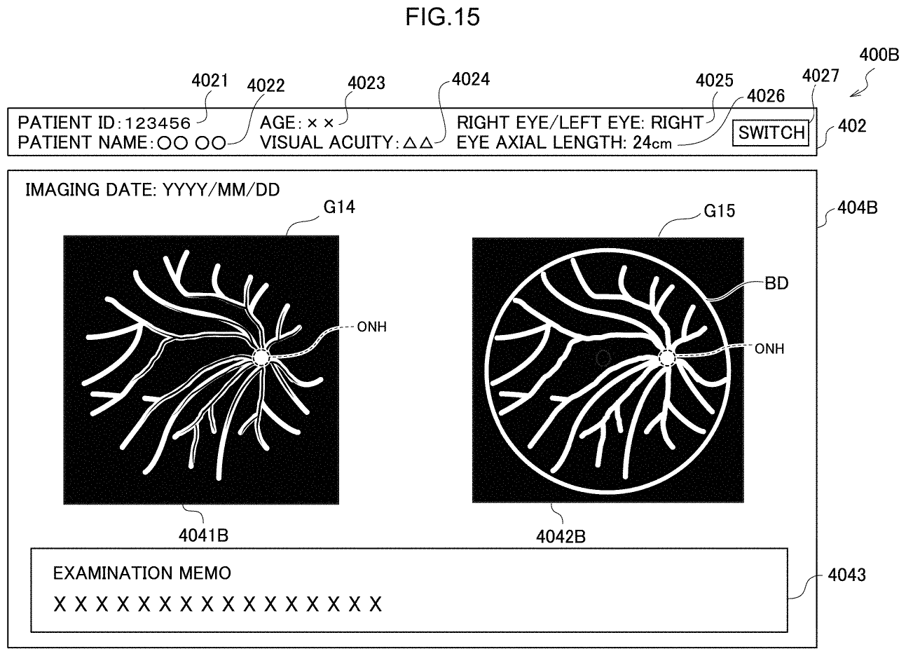

When the switch screen icon 4027 is operated in a state in which the original fundus image UWFGP and the blood vessel extraction image G4 are being displayed in the image display area 404A, the examination screen 400A is changed to an examination screen 400B illustrated in FIG. 15. The content is similar in the examination screen 400A and the examination screen 400B, and so the same reference numerals are appended to parts with similar content, explanation thereof will be omitted, and explanation will be of the differing parts of the content alone.

As illustrated in FIG. 15, the examination screen 400B includes a combined image display field 4041B and a separate blood vessel extraction image display field 4042B instead of the original fundus image display field 4041A and the blood vessel extraction image display field 4042A of FIG. 14. A combined image G14 is displayed in the combined image display field 4041B. A processing image G15 is displayed in the blood vessel extraction image display field 4042B.

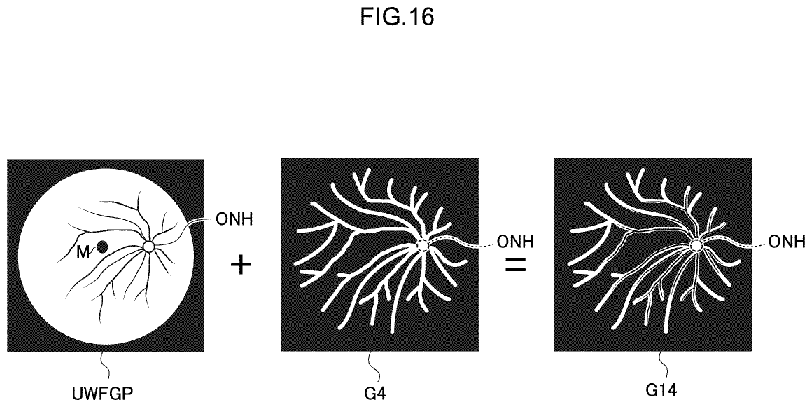

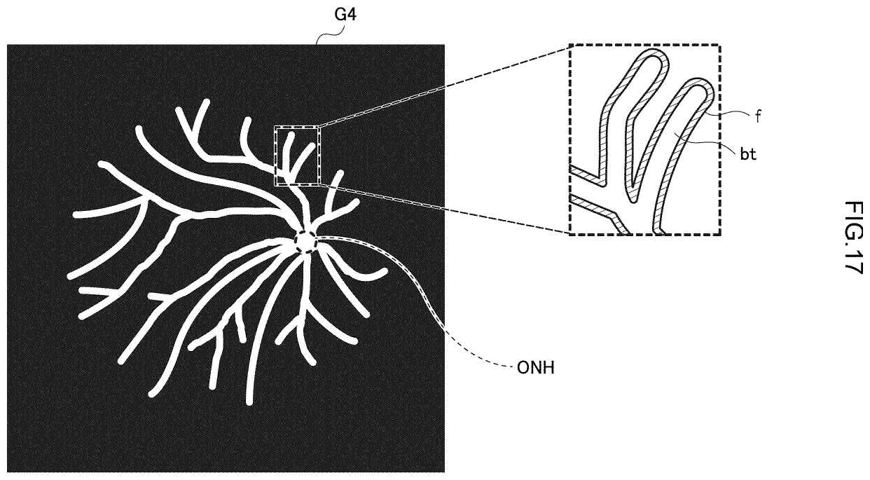

The combined image G14 is an image in which the blood vessel extraction image G4 is overlaid on the RG color fundus image UWFGP (original fundus image), as illustrated in FIG. 16. A user is easily able to ascertain a state of the choroidal blood vessels on the RG color fundus image UWFGP (original fundus image) using the combined image G14.

The processing image G15 is an image in which the boundary BG is displayed overlaid on the blood vessel extraction image G4 by appending a frame (boundary line) indicating the boundary BD between the background area BG and the foreground area FG to the blood vessel extraction image G4. A user is able to easily discriminate between the fundus region and a background area using the processing image G15 in which the boundary BD is displayed overlaid.

Note that the blood vessel extraction image G4 in the blood vessel extraction image display field 4042A of FIG. 14, and the processing image G15 in the separate blood vessel extraction image display field 4042B of FIG. 15 may have further emphasis of the choroidal blood vessels by applying a frame f to the blood vessel bt as illustrated in FIG. 17.

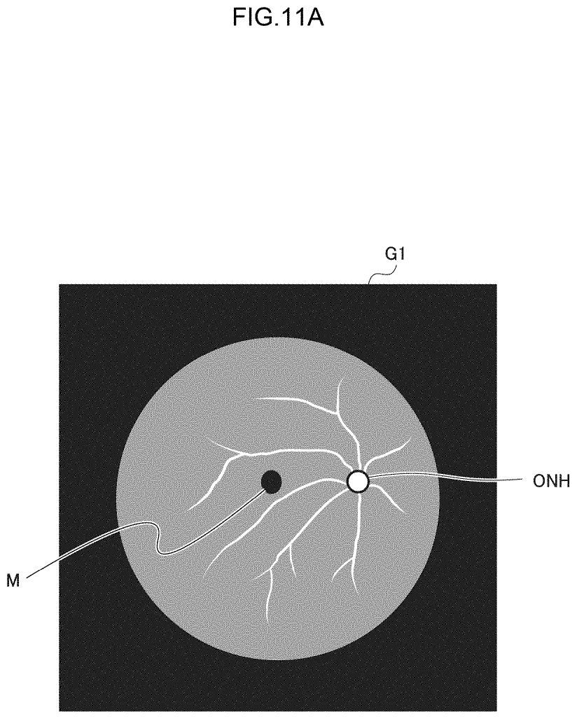

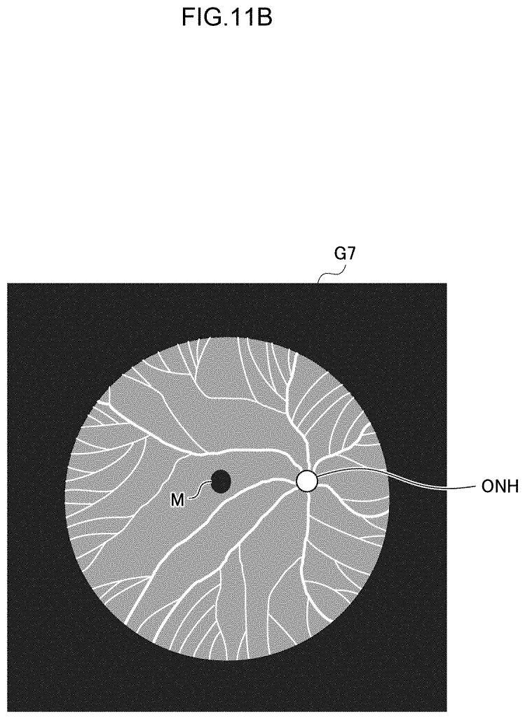

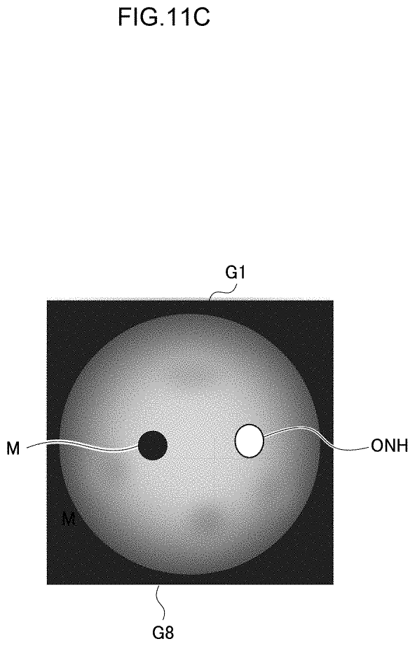

Hitherto a blood vessel emphasis image G7 as illustrated in FIG. 11B was obtained from the choroidal vascular image G1 illustrated in FIG. 11A, and each of the pixels of the foreground area image of the blood vessel emphasis image G7 binarized using an average value of pixel values for a specific number of pixels centered on each respective pixel as the threshold value. The threshold value in such cases is a low value in a peripheral portion of the foreground area image as illustrated in FIG. 11C. This is because at the outside of pixels of the peripheral portion of the foreground area image there are pixels present in the background area image having pixel values of zero, and the zero values lower the value of the average value. This means that the threshold value for the peripheral portion of the blood vessel emphasis image G7 is set low due to being influenced by the pixel values of the background area image (=0), and a frame (white portion) occurs in a peripheral portion of the foreground area in a blood vessel extraction image G9 obtained by such binarization, as illustrated in FIG. 11D. Thus there is a concern that the frame occurring at the peripheral portion of the foreground area FB of blood vessel extraction image G9 might be mistakenly extracted as a blood vessel, and the user (ophthalmologist) might be caused to recognize blood vessels as being present in a portion of the foreground area FB where there are not actually blood vessels present.

To address this issue, in the present exemplary embodiment the background processing complete image G2 (see FIG. 10B) is generated in which pixel values are infilled based on the background area BG image and the foreground area FG image of the choroidal vascular image G1 illustrated in FIG. 10A. Binarization from the background processing complete image G2 is via the blood vessel emphasis processing, and so a frame (white portion) does not occur in the peripheral portion of the blood vessel extraction image G4, as illustrated in FIG. 10D. The present exemplary embodiment is accordingly able to prevent the boundary between the foreground area and the background area from affecting the results of analyzing the fundus image. The present exemplary embodiment is accordingly able to prevent the user (ophthalmologist) from recognizing choroidal blood vessels as being present in portions where blood vessel are not actually present in the blood vessel extraction image G4 (namely in the background area, the outermost peripheral portion of the foreground area, and the like).

Binarization of the blood vessel emphasis image G3 described above is performed for each of the pixels of the foreground area FG using the average value H of pixel values of the specific number of pixels centered on the respective pixel as the threshold value, however the technology disclosed herein is not limited thereto, and the following modified examples of binarization processing may be employed.

### Modified Example 1 of Binarization Processing

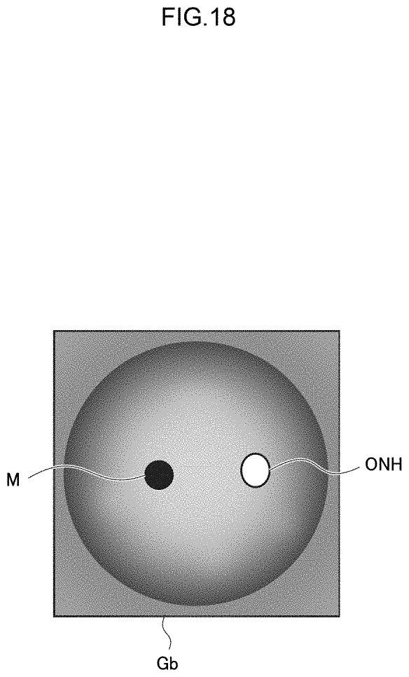

By blurring the blood vessel emphasis image G3 (for example by performing processing to remove low frequency components from the image), the fundus image processing section 2060 generates a blurred image Gb illustrated in FIG. 18 and then uses pixel values of each of the pixels of the blurred image Gb as the threshold value for each of the pixels of the blood vessel emphasis image G3 corresponding to the positions of each of the pixels of the blurred image Gb. An example of processing to blur the blood vessel emphasis image G3 is convolution computation using a point spread function (PSF) filter. Filtering processing using a Gaussian filter, a low pass filter, and the like may also be used as the processing to blur the image.

### Modified Example 2 of Binarization Processing

The fundus image processing section 2060 may employ a predetermined value as the threshold value for binarization processing. Note that the predetermined value is, for example, an average value of all the pixel values of the foreground area FG.

### Modified Example 3 of Binarization Processing

A Modified Example 3 of binarization processing is an example in which step 302 of FIG. 6 (steps 332 to 342) is omitted. In such cases the content of processing for step 356 of FIG. 9 is as follows.

First the fundus image processing section 2060 extracts a specific number of pixels centered on a pixel m.

The fundus image processing section 2060 determines whether or not there is a pixel of the background area BG contained in the specific number of pixels extracted.

In cases in which determination is that there is a pixel of the background area BG contained in the specific number of pixels extracted, the fundus image processing section 2060 replaces the pixel of the background area BG with the following pixel, and sets pixels of the foreground area including the replacement pixel and the pixels initially extracted as the specific number of pixels centered on the pixel m. The pixel to replace the background area BG pixel is a pixel of the foreground area FG adjacent to the pixels of the foreground area FG contained in the specific number of pixels (a pixel of the foreground area image positioned at only a specific distance from each of the pixels).

However, when determined that there is no background area BG pixel contained in the specific number of pixels extracted, the fundus image processing section 2060 does not perform the pixel replacement described above, and sets the pixels initially extracted as the specific number of pixels centered on the pixel m.

In other words, in the Modified Example 3 of binarization processing the following image processing step is executed by the fundus image processing section 2060. Acquisition is performed to acquire a fundus image including a foreground area that is an image portion of the examined eye and a background area to the image portion of the examined eye. Next, binarization is performed on the pixel values of each of the pixels of the foreground area image based on only the pixel values of pixels of the foreground area image positioned a specific distance from the respective pixel.

### Other Modified Examples

In the exemplary embodiment described above, the pixel values of the background area are a value of black, i.e. zero, in the detectors 70, 72, 74, 76, however technology disclosed herein is not limited thereto, and a configuration may be employed in which the pixel values of the background area are a value of white.

Although a fundus image (UWF-SLO image (for example, UWFGP (see FIG. 3)) is acquired by the ophthalmic device 110, a fundus image (FCGQ (see FIG. 3)) may be acquired using a fundus camera. In cases in which a fundus image FCGQ is acquired using a fundus camera, the R component, the G component, and the B component of RGB space is employed in the image processing described above. Note that the a* component of L*a*b* space may be employed, or another component of another space may be employed.

In the technology disclosed herein, the image processing illustrated in FIG. 6 is not limited to being executed by the server 140, and may be executed by a separate computer connected to the ophthalmic device 110, the viewer 150, or the network 130.

Moreover, although the ophthalmic device 110 includes functionality to image a region having an internal illumination angle of 200° with respect to a position of the eyeball center O of the examined eye 12 (an external illumination angle of 167° with respect to the pupil of the eyeball of the examined eye 12), there is no limitation to this angle. The internal illumination angle may be 200° or greater (an external illumination angle of from 167° to 180°).

Furthermore, a specification may be employed in which the internal illumination angle is less than 200° (the external illumination angle is less than 167°). The following angles of view may, for example, be employed: an internal illumination angle of about 180° (an external illumination angle of about 140°), an internal illumination angle of about 156° (an external illumination angle of about 120°), an internal illumination angle of about 144° (an external illumination angle of about 110°). These numerical values are merely examples.

Although explanation has been given in the examples described above regarding examples in which a computer is employed to implement image processing using a software configuration, the technology disclosed herein is not limited thereto. For example, instead of the image processing being executed by a software configuration employing a computer, the image processing may be executed solely by a hardware configuration such as a field programmable gate array (FPGA) or an application specific integrated circuit (ASIC). Alternatively, a configuration may be adopted in which some processing out of the image processing is executed by a software configuration, and the remaining processing is executed by a hardware configuration.

Such technology disclosed herein encompasses cases in which the image processing is implemented by a software configuration utilizing a computer, and also image processing implemented by a configuration that is not a software configuration utilizing a computer, and encompasses the following first technology and second technology.

First Technology

An image processing device including:

an acquisition section configured to acquire a first fundus image of an examined eye including a foreground area and a background area other than the foreground area; and

a generation section configured to generate a second fundus image by the processor performing background processing to replace a first pixel value of a pixel configuring the background area with a second pixel value different from the first pixel value.

The fundus image processing section 2060 of the exemplary embodiment described above is an example of an “acquisition section” and a “generation section” of the first technology above.

Second Technology

An image processing method including:

an acquisition section acquiring a first fundus image of an examined eye including a foreground area and a background area other than the foreground area; and

a generation section generating a second fundus image by the processor performing background processing to replace a first pixel value of a pixel configuring the background area with a second pixel value different from the first pixel value.

The following third technology is proposed from the content disclosed above.

Third Technology

A computer program product for image processing, the computer program product including a computer-readable storage medium that is not itself a transitory signal, with a program stored on the computer-readable storage medium, the program causing a computer to execute processing including:

acquiring a first fundus image of an examined eye including a foreground area and a background area other than the foreground area; and

generating a second fundus image by performing background processing to replace a first pixel value of a pixel configuring the background area with a second pixel value different from the first pixel value.

It must be understood that the image processing described above is merely an example thereof. Obviously redundant steps may be omitted, new steps may be added, and the processing sequence may be swapped around within a range not departing from the spirit of technology disclosed herein.

All publications, patent applications and technical standards mentioned in the present specification are incorporated by reference in the present specification to the same extent as if each individual publication, patent application, or technical standard was specifically and individually indicated to be incorporated by reference.

1. An image processing method comprising:
a processor acquiring a first fundus image of an examined eye including a foreground area and a background area other than the foreground area; and
the processor generating a second fundus image by performing background processing to replace a first pixel value of a pixel configuring the background area with a second pixel value different from the first pixel value.

2. The image processing method of claim 1, wherein the foreground area is an area in which a specific area of the examined eye is imaged.

3. The image processing method of claim 2, wherein the specific area is a fundus region of the examined eye.

4. The image processing method of claim 1, wherein the background area is a single color area.

5. The image processing method of claim 1, further comprising the processor generating a third fundus image by binarizing pixel values of pixels of the foreground area in the second fundus image or in an image resulting from emphasizing blood vessels in the second fundus image, by binarization with respect to a threshold value determined based on pixel values of peripheral pixels to the pixels of the foreground area.

6. The image processing method of claim 1, further comprising the processor executing processing to analyze blood vessels of a fundus of the examined eye.

7. The image processing method of claim 6, wherein the blood vessels are choroidal blood vessels.

8. The image processing method of claim 1, further comprising the processor replacing, with respect to the second fundus image, a pixel value of a pixel of the background area with a third pixel value different from the second pixel value.

9. The image processing method of claim 8, wherein the first pixel value is the same as the third pixel value.

10. The image processing method of claim 1, wherein the background processing is performed on at least pixels adjacent to pixels of the foreground area, among pixels configuring the background area.

11. The image processing method of claim 1, wherein the second pixel value is a value in a range of possible values for pixel values of pixels in the foreground area.

12. The image processing method of claim 1, wherein the second pixel value is a pixel value of a pixel, among pixels in the foreground area, which is closest in distance to the pixel of the background area, or is an average value of pixel values of pixels in the foreground area.

13. An image processing device comprising:
a memory, and
a processor coupled to the memory,
wherein the processor:
acquires a first fundus image of an examined eye including a foreground area and a background area other than the foreground area; and
generates a second fundus image by performing background processing to replace a first pixel value of a pixel configuring the background area with a second pixel value different from the first pixel value.

14. A non-transitory storage medium storing a program that causes a computer to execute processing comprising:
acquiring a first fundus image of an examined eye including a foreground area and a background area other than the foreground area; and
generating a second fundus image by performing background processing to replace a first pixel value of a pixel configuring the background area with a second pixel value different from the first pixel value.
